(19) United States
(12) Patent Application Publication
(10) Pub. No.: US 2023/0404643 A1
(43) Pub. Date: Dec. 21, 2023

(54) SYSTEMS, APPARATUSES, AND METHODS FOR MEDICAL DEVICES

(71) Applicant: Boston Scientific Scimed, Inc., Maple Grove, MN (US)

(72) Inventor: Patrick NICOLAS, Mansfield, MA (US)

(73) Assignee: Boston Scientific Scimed, Inc., Maple Grove, MN (US)

(21) Appl. No.: 18/334,517

(22) Filed: Jun. 14, 2023

Related U.S. Application Data
(60) Provisional application No. 63/366,539, filed on Jun. 17, 2022.

(51) Int. Cl. A61B 18/04

(52) U.S. Cl. CPC A61B 18/04; A61B 2018/00547

(57) ABSTRACT

A medical device may comprise a handle, which may include a grip portion; a body portion; and a ram housing extending proximally of the body portion; and a shaft having a first, proximal portion and a second, distal portion. The first portion of the shaft may include a first outer sheath. The second portion of the shaft may include a second outer sheath. The first outer sheath may be more rigid than the second outer sheath. The medical device may further comprise a rigid ram extending through at least a portion of the body portion and at least a portion of the shaft. The ram may be configured to be transitioned from a first, proximal configuration, in which the second portion of the shaft is flexible, and a second, distal configuration, in which the second portion of the shaft is rigid.

### CROSS-REFERENCE TO RELATED APPLICATION(S)

This patent application claims the benefit of priority under 35 U.S.C. § 119 to U.S. Provisional Patent Application No. 63/366,539, filed Jun. 17, 2022, which is herein incorporated by reference in its entirety.

### TECHNICAL FIELD

The present disclosure relates generally to devices, systems, and methods for medical devices and, in particular, to devices, systems, and methods for transitioning shafts of medical devices from flexible configurations to rigid configurations.

### BACKGROUND

Certain medical conditions, such as conditions of the prostate, may be treated by ablation, including by vapor ablation. Such ablation may be performed using a device having a sheath that is inserted into a body lumen or otherwise into a body of a subject (e.g., patient). Certain devices may include a rigid, straight shaft that is inserted into, for example, a urethra of a subject and advanced through the urethra to the prostatic urethra (a portion of the urethra passing through the prostate). During insertion, the rigid, straight shaft must navigate curved portions of the urethra carefully, so as to avoid causing a perforation. Therefore, use of the device may require high amounts of skill and increased amounts of procedure time to appropriately navigate the device. A rigid shaft may cause discomfort to a subject during insertion.

The systems, devices, and methods of the current disclosure may rectify some of the deficiencies described above, and/or address other aspects of the prior art.

### SUMMARY

A medical device may comprise a handle, which may include a grip portion; a body portion; and a ram housing extending proximally of the body portion; and a shaft having a first, proximal portion and a second, distal portion. The first portion of the shaft may include a first outer sheath. The second portion of the shaft may include a second outer sheath. The first outer sheath may be more rigid than the second outer sheath. The medical device may further comprise a rigid ram extending through at least a portion of the body portion and at least a portion of the shaft. The ram may be configured to be transitioned from a first, proximal configuration, in which the second portion of the shaft is flexible, and a second, distal configuration, in which the second portion of the shaft is rigid.

Any of the medical devices disclosed herein may include any or all of the following features. A proximal end of the ram may protrude from a proximal end of the body portion of the handle. The ram may be transitioned from the first configuration to the second configuration by moving the proximal end of the ram distally. The proximal end of the ram may include a tab. The proximal end of the ram may be received within the ram housing. The tab may extend through a slot in the ram housing. In the second configuration, the tab may abut a proximal surface of the body portion of the handle. An axis of the grip portion may be transverse to a longitudinal axis of the first portion of the shaft. In the first configuration, a distal-most end of the ram may be proximal of at least a portion of the second portion of the shaft. The first outer sheath may be approximately as rigid as a distal portion of the ram. At least a portion of the ram may have a cross-sectional shape that is approximately a segment of a circle or approximately an arc of a circle. At least the portion of the ram may include a curved, radially outward-facing surface, and an approximately planar, radially inward-facing surface. A guide, having a first arm and a second arm, may extend radially inwardly from the approximately planar, radially inward-facing surface. A lumen may be received between the first arm and the second arm. A needle may be received within the lumen. A distal opening of the needle may be in fluid communication with a vapor generator in the handle. The second portion of the shaft may include a flexible, tubular sheath.

In another example, a medical device may comprise a handle; a shaft having a first portion, having a rigid outer sheath, and a second portion, having a flexible outer sheath. Each of the first portion and the second portion of the shaft may be configured to be inserted into a body lumen of a subject. The medical device may also comprise a rigid ram that is configured to be transitioned from a first configuration, in which a distal-most end of the ram is proximal of the second portion of the shaft, to a second configuration, in which the distal-most end of the ram is within the second portion of the shaft. A proximal end of the ram may protrude from a proximal end of the handle, and the ram may be transitioned from the first configuration to the second configuration by moving the proximal end of the ram in a distal direction.

Any of the medical devices disclosed herein may include any or all of the following features. The second portion of the shaft may be bendable in the first configuration, and the second portion of the shaft is rigid in the second configuration. The proximal end of the ram is received within a ram housing. A tab of the proximal end of the ram may extend through a slot in the ram housing.

An exemplary medical method may comprise inserting a distal end of a shaft of a medical device into a body lumen of a subject; navigating the distal end of the shaft to a treatment location; and, after navigating the distal end of the shaft to the treatment location, moving a ram distally from a first configuration to a second configuration. A portion of shaft may be bendable in the first configuration. The portion of the shaft may be rigid in the second configuration. The method may further include, in the second configuration, extending a needle from a distal tip of the shaft; and delivering a vapor from the needle.

Any of the methods disclosed herein may include any or all of the following aspects. The body lumen may be a urethra. The vapor may be configured to ablate at least a portion of a prostate of the subject.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” As used herein, the term “proximal” means a direction closer to an operator and the term “distal” means a direction further from an operator. Although vapor ablation devices may be referenced herein, such references should not be construed as limiting. The examples disclosed herein may also be used with other types of ablation mechanisms (e.g., cryoablation, RF ablation, or other types of ablation) or with other devices not relating to ablation. The terms approximately, substantially, and the like mean +/−10% from the stated value or other characteristic.

### BRIEF DESCRIPTION OF THE DRAWINGS

The accompanying drawings, which are incorporated in and constitute a part of this specification, illustrate examples of the present disclosure and together with the description, serve to explain the principles of the disclosure.

FIGS. 1A and 1B depict side views of an exemplary medical device.

FIGS. 2A and 2B depict partially schematic side views of another exemplary medical device.

FIG. 2C depicts a partially transparent perspective view of a portion of the exemplary medical device of FIGS. 2A and 2B.

FIG. 3 depicts a partially schematic view of a distal end of any of the medical devices of FIGS. 1A-2B.

FIGS. 4-6 depict alternative medical devices.

### DETAILED DESCRIPTION

A medical device, such as a medical device for vapor ablation of a prostate, may include a handle and a shaft extending distally from the handle. At least a portion of the shaft may include a flexible sheath. The handle of the device may have a pistol-type grip, such that a portion of the handle that is gripped by a user extends transversely from a longitudinal axis of the shaft. A movable ram may extend parallel or coaxial with the longitudinal axis of the shaft, through at least a portion of the handle and through at least a portion of the shaft. A proximal end of the ram may protrude proximally out of the handle. In a first configuration, a distal end of the ram may be in a first, proximal position, such that the distal portion of the shaft is flexible and can be bent. In a second configuration, a distal end of the ram may be in a second, distal position, such that the ram stiffens at least a distal portion of the shaft. To transition between the first and the second configurations, the user may advance or retract the portion of the ram that protrudes proximally from the handle. Although the Figures and the description below describe different aspects of exemplary devices, the features of any of the devices disclosed herein may be combined in any manner, unless stated otherwise.

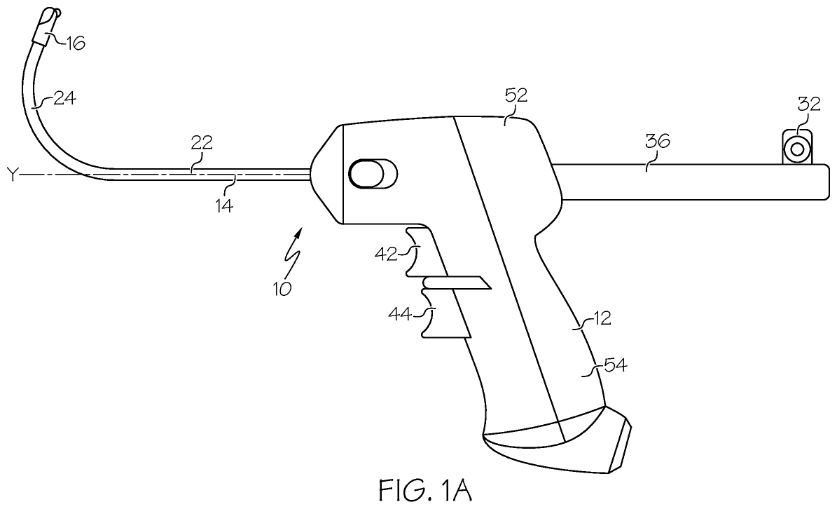

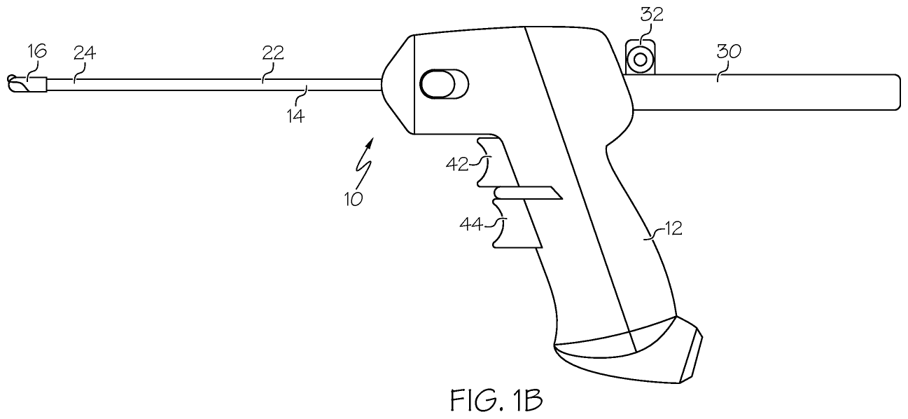

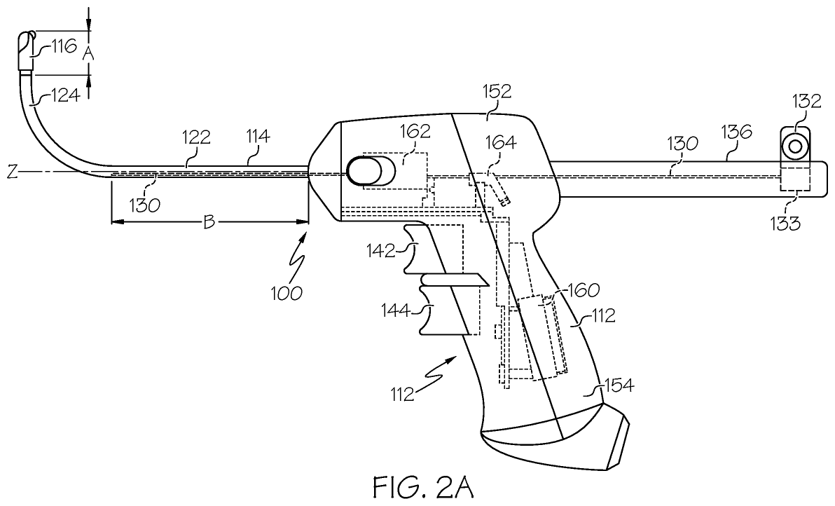

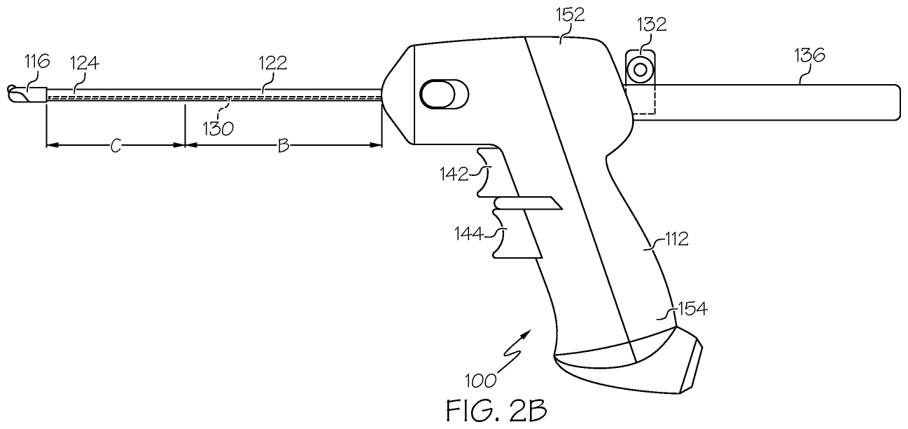

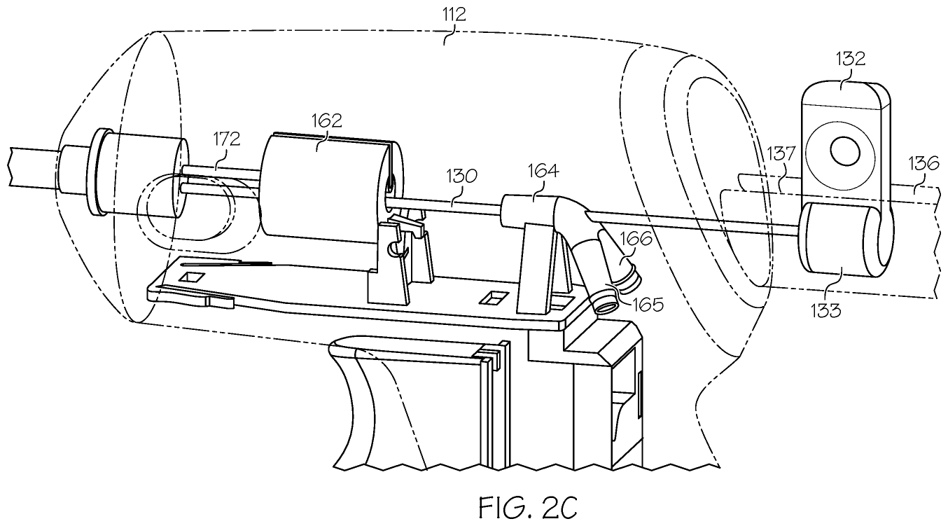

FIGS. 1A and 1B show side views of an exemplary medical device 10 in a first configuration (FIG. 1A) and a second configuration (FIG. 1B). Medical device 10 (also referred to herein as device 10) (or any of the other medical devices disclosed herein) may include a duodenoscope, endoscope, gastroscope, endoscopic ultrasonography (“EUS”) scope, colonoscope, ureteroscope, bronchoscope, laparoscope, cystoscope, aspiration scope, sheath, catheter, or other device having a shaft for insertion into a body lumen or other opening of a body. Medical device 10 may include a medical tool such as, for example, an ablation device. Device 10 may include a handle 12 and a shaft 14, which may be insertable into a body lumen of a subject or otherwise into a body of a subject (e.g., through a tissue of a subject, such as via a transperineal route or laparoscopic route). Device 10 may also include a ram (not shown in FIGS. 1A and 1B but having any of the properties of a ram 130, depicted in FIGS. 2A and 2B, and described below), that may be movable between first (proximal) and second (distal) configurations, as discussed below.

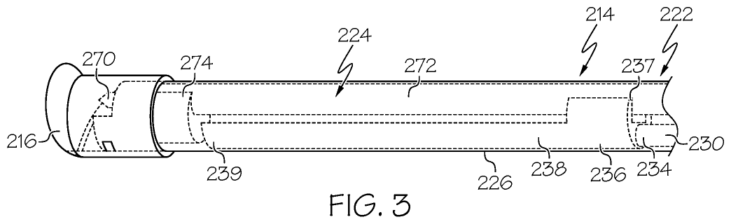

Shaft 14 may have a distal tip 16. Distal tip 16 may have an atraumatic shape (e.g., a rounded, bulbous shape) and may be made of any suitable material, including a plastic material, which may be transparent (e.g., acoustically transparent). Distal tip 16 may alternatively be made of metal, resin, Ultem®, polyurethane, or other materials, or a combination of materials. A needle, discussed further below with respect to FIG. 3, may extend through a lumen of shaft 14 and through an opening of distal tip 16. Other lumens, cables, wires, and the like may also extend through shaft 14 and/or distal tip 16.

Shaft 14 may include a proximal portion 22 and a distal portion 24. Distal portion 24 may extend between a distal end of proximal portion 22 and a proximal end of distal tip 16. Distal portion 24 may include flexible components, such that distal portion 24 may be bendable in some configurations, as discussed below. For example, distal portion 24 may include a flexible, tubular, outer sheath, flexible lumen(s), a flexible needle, and flexible cables/wires. Shaft 14 may have any of the properties of any other shafts described herein, such as a shaft 214 (see FIGS. 2A-B), discussed below. Some (e.g., distal portion 24) of shaft 14 or all of shaft 14 may include materials such as polytetrafluoroethylene (“PTFE”), thermoplastic elastomer (“TPE”), nylon(s), Pebax®, or polyethylene urethane. In some configurations, distal portion 24 may be passively bendable, such that, when shaft 14 is inserted into a body lumen, shaft 14 may bend as it encounters body structures. For example, shaft 14 may bend as it is passed through curved portions of a subject's urethra. In alternatives, distal portion 24 may be articulable by a user, via one or more actuators of handle 12 connected to wires or cables that are fixed or otherwise operably coupled to distal portion 24.

Proximal portion 22 of shaft 14 may be rigid. For example, proximal portion 22 may be rigid in all configurations of device 10/shaft 14. For example, proximal portion 22 may include a rigid, tubular, outer sheath. Proximal portion 22 may include any suitable material, such as, for example, plastic or metal. In alternatives, proximal portion 22 may include flexible components and may include the same or different materials as distal portion 24.

Shaft 14 may be sized and shaped so as to fit within a body lumen of a subject. For example, shaft 14 may have a length and width to accommodate a urethra. Shaft 14 may have a uniform diameter or a variable diameter. For example, shaft 14 may have a diameter that is approximately 20 French or approximately 10-30 French. Shaft 14 (including distal portion 24 and proximal portion 22) may have any suitable length dimensions, such as the length dimensions described below, with respect to a shaft 114 of device 100.

Handle 12 may have an approximately pistol shape, with a housing 52 extending approximately parallel to or coaxial with a central longitudinal axis Y of proximal portion 22 of shaft 14. Handle 12 may also include a grip portion 54, extending transversely to housing 52. Handle 12 (such as grip portion 54 of handle 12) may include a plurality of actuators, such as buttons 42 and 44, for controlling aspects of device 10. For example, button 44 may control a delivery of fluids (e.g., saline) through handle 12 and shaft 14. For example, a user may press button 42 to provide a fluid flush and/or to control deployment of the needle that extends through a lumen of shaft 14. For example, button 42 may include a two-stage button, such that depressing button 42 part-way delivers a fluid flush, and fully depressing button 42 causes the needle to deploy. Button 44 may control delivery of vapor, or another substance, through the needle. For example, a user may press button 42 partially to provide a flush and then further depress button 42 to cause a distal tip of the needle to extend from distal tip 16 of shaft 14. The needle may extend radially outwardly (e.g., transversely) from shaft 14 and may have a lumen extending therethrough to deliver vapor or other fluids through one or more openings at or near a distal-most end of the needle. The user may then press button 44 to deliver vapor or another substance, in order to ablate tissue (e.g., prostate tissue) near the needle.

A ram (not shown in FIGS. 1A and 1B) may extend through at least a portion of shaft 14 and at least a portion of housing 52 of handle 12, and protrude proximally from a proximal end of housing 52. The ram may have any features of a ram 130, described below with respect to FIGS. 2A and 2B. A ram housing 36 may extend proximally from handle 12, and may receive a proximal portion of the ram. A tab 32 may be fixed to a proximal-most end (or another portion) of the ram. Tab 32 may extend radially outward from the ram. Ram housing 36 may include a slot or other opening extending longitudinally along ram housing 36, such that tab 32 may extend through the slot to protrude radially outwardly from ram housing 36. Ram housing 36 may be approximately the same length as distal portion 24. The ram, tab 32, and/or ram housing 36 may include one or more locks to retain the ram in a selected position. Such locks may include, for example, levers, knobs, slots, or other features and may operate according to frictional forces or by blocking portions of the slot extending longitudinally along ram housing 36.

As shown in FIGS. 1A and 1B, the ram may be utilized to transition device 10 from a first configuration, in which distal portion 24 of shaft 14 is flexible, to a second configuration, in which distal portion 24 of shaft 14 (and proximal portion 22 of shaft 14) is rigid. In the first configuration (FIG. 1A), the ram may be in a first, proximal configuration (e.g., a retracted configuration). In the second configuration (FIG. 1B), the ram may be in a second, distal configuration (e.g., extended configuration). The first configuration may be utilized during insertion of shaft 14 into a body lumen, such as a urethra, of a subject. Because distal portion 24 is flexible in the first configuration, it may be able to navigate curved portions of the subject's anatomy. Flexibility of distal portion 24 may make insertion easier, less time consuming, less subject to accidental perforations through the body lumen, and more comfortable for the subject. As discussed below, an operator may utilize the ram to transition device 10 to the second configuration (FIG. 1B). For example, the operator may transition device 10 to the second configuration after shaft 14 is positioned within the body lumen. In examples where device 10 is a vapor ablation device, vapor may be delivered to the prostate through the needle when the device is in the second (rigid) configuration. The vapor may be configured to ablate at least a portion of the prostate. In the rigid configuration, device 10 may have characteristics similar to that of devices that have a shaft that is always rigid. Delivering treatments with device 10 in the second configuration may provide familiarity to operators who are already familiar with devices having rigid shafts. Thus, the operators may not require extensive retraining and may provide performance akin to a device having an entirely stiff shaft.

In the first configuration (FIG. 1A), tab 32 may be at or near a proximal end of ram housing 36, and the ram may be in a proximal configuration, such that a distal-most end of the ram is at or near a proximal end of distal portion 24 of shaft 14. Because the ram does not extend through distal portion 24, distal portion 24 may be flexible and able to passively bend as distal portion 24 moves through an anatomy of a subject. For example, distal portion 24 may bend as it passes through tortuous portions of a body lumen, such as a urethra. In the first configuration, distal portion 24 may have sufficient columnar force such that it may be advanced through the anatomy of the subject (e.g., through a body lumen of the subject). A lock may be activated to retain the ram at in the first configuration.

Materials of distal portion 24 may be selected so as to provide a desired radius of curvature of distal portion 24. Any suitable radius of curvature may be chosen. The radius of curvature may correspond to a shape of a subject's anatomy. For example, a radius of curvature of distal portion 24 when it is bent may correspond to a curvature of a portion of the urethra, such as the membranous urethra, which passes through the pelvic floor and into the penis, between the prostatic urethra and the spongy urethra. As shown in FIG. 1A, distal portion 24 may be bendable such that distal tip is 16 is deflected from a longitudinal axis Y of proximal portion 22 by more than 90 degrees.

In the second configuration (FIG. 1B), tab 32 may be at or near a distal end of ram housing 36, and the ram may be in a distal configuration, such that a distal-most end of the ram is at or near distal tip 16. Because the ram extends through distal portion 24, distal portion 24 may be more rigid than in the first configuration (FIG. 1). For example, in the second configuration, an entirety of shaft 14 (including proximal portion 22 and distal portion 24) may have a uniform rigidity. In other words, in the second configuration, distal portion 24 may be as rigid as proximal portion 22. The second configuration of FIG. 1B may be utilized while delivering treatments at a desired treatment location. In the second configuration, distal portion 24 and proximal portion 22 may be approximately coaxial, regardless of forces exerted on distal portion 24 by a subject's anatomy. A lock may be activated to retain the ram in the second configuration.

In use, an operator may perform a medical method, including inserting a distal tip of shaft 14 into a urethra of a subject in the first, flexible configuration (FIG. 1A) and advancing distal tip 16 distally within the urethra. After shaft 14 is positioned such that distal tip 16 is within the prostatic urethra and in a desired location to deliver treatment, the operator may transition the device to the second, rigid configuration (FIG. 1B). To transition device 10 to the second configuration, the operator may move or otherwise slide tab 32 and the ram distally, relative to ram housing 36, handle 12, and shaft 14. In the second configuration, tab 32 may abut a proximal end of housing 52 of handle 12, such that housing 52 acts as a stop for tab 32 (and the ram). The distal end of the ram may extend to or near to distal tip 16 and a distal end of distal portion 24. During extension or advancement of the ram relative to shaft 14, the ram stiffens distal portion 24, such that it becomes rigid. With shaft 14 in the second configuration, the operator may deliver a treatment to the subject. For example, the user may depress button 42 in order to advance a needle and button 44 in order to transurethrally deliver vapor to a prostate of the subject. An operator also may transition device 10 from the second (rigid) configuration to a first (flexible) configuration during a procedure. For example, the operator may move or otherwise slide tab 32 and the ram proximally, relative to ram housing 36, handle 12, and shaft 14. The operator may then move a position of distal tip 16, deliver further treatment, and/or withdraw shaft 14 from the body lumen.

An operator may additionally or alternatively slide tab 32 distally or proximally to positions between the proximal end of ram housing 36 and the distal end of ram housing 36, such that the distal end of the ram is within distal portion 24 of shaft 14 (between a proximal end of distal portion 24 and a proximal end of distal tip 16). A portion of distal portion 24 that is distal to the distal end of the ram may be relatively more flexible than a portion of distal portion 24 that is proximal to the distal end of the ram. In other words, the ram may extend partially through distal portion 24, with a portion of distal portion 24 through which the ram extends being relatively more rigid than a portion of distal portion 24 through which the ram does not extend. Ram housing 36 may include markings or intermediate stops or locks to allow an operator to adjust the ram to a predefined position within distal portion 24.

Thus, device 10 may provide for a desired flexibility during insertion, while providing a stiffness that is familiar to operators who have used existing devices during delivery of the treatment. An operator may also opt to operate device 10 only in the second configuration. In such a scenario, an operator may activate a lock to retain the ram and other elements of device 10 in the rigid configuration. An operator may prefer to insert shaft 14 into a body lumen with shaft 14 in the rigid configuration (FIG. 1B). For example, the operator may prefer to use the rigid configuration due to operator preference, an anatomy of the subject, a type of treatment, or any other reason. Device 10 may thus provide adaptability in use, such that different operators may use device 10 subject to their own preferences. Device 10 thus may help institutions avoid buying multiple different types of devices to satisfy preferences of different operators.

FIGS. 2A and 2B show a device 100 in a first, flexible configuration (FIG. 2A) and a second, rigid configuration (FIG. 2B). FIG. 2C shows a partially-transparent, perspective view of a portion of device 100. Except as where specified, device 100 may have any of the properties of device 10, described above. Where possible, reference numbers of FIGS. 2A-2C add 100 to like structures of FIGS. 1A and 1B.

Device 100 may include a handle 112 and a shaft 114. Shaft 114 may include a distal tip 116. Handle 112, shaft 114, and distal tip 116 may have any of the properties of handle 12, shaft 14, and distal tip 16, respectively, described above. Handle 112 may include buttons 142, 144, which may have any of the properties of buttons 42, 44, respectively, discussed above.

Shaft 114 may include a proximal portion 122 and a distal portion 124. Proximal portion 122 may have any of the features of proximal portion 22, described above. Distal portion 124, like distal portion 24, may include flexible materials and any of the same features. Shaft 114 may have any suitable length. For example, a total length of shaft 114 (including distal tip 116) may be approximately 23.12 cm, or a total length of approximately 15 cm to approximately 30 cm. A length “A” of distal tip 116 may be approximately 2.8 cm, or a length “A” of distal tip 116 may be approximately 2.5 cm to approximately 3 cm. A length “B” of proximal portion 122 may be approximately 12.7 cm, or a length “B” of proximal portion 122 may be approximately 10 cm to approximately 15 cm. A length “C” (see FIG. 2B) of distal portion 124 may be approximately 7.62 cm, or a length “C” of distal portion 124 may be approximately 2 cm to approximately 112 cm. The above dimensions are merely exemplary, and other dimensions may be utilized. For example, dimensions of portions of shaft 114 may vary for medical devices 100 configured for use in different types or sizes of anatomy.

Materials of distal portion 124 may be selected so as to provide a desired radius of curvature of distal portion 124 when distal portion 124 is in the first configuration. Any suitable radius of curvature may be chosen. The radius of curvature may correspond to a shape of a subject's anatomy. For example, a radius of curvature of distal portion 124 when it is bent may be approximately 5 cm, or approximately 3 m to approximately 7 cm, corresponding to a curvature of a portion of the urethra, such as the membranous urethra, which passes through the pelvic floor and into the penis, between the prostatic urethra and the spongy urethra. As shown in FIG. 2A, distal portion 124 may be bendable in the first configuration such that distal tip is 116 is deflected from a longitudinal axis of proximal portion 122 by approximately 90 degrees. As compared to device 10, a distal tip of device 100 may deflect by a lesser amount.

FIGS. 2A and 2B show a ram 130, extending through a ram housing 136, which protrudes proximally from a proximal end of handle 112. A proximal end of ram 130 may include a body 133, which may be fixed to a tab 132. Body 133 may include or be similar to a plunger. For example, body 133 may have an approximately cylindrical shape, with a wider radius/diameter than ram 130. Body 133 may facilitate linear, longitudinal movement of ram 130, without ram 130 bending or otherwise deflecting. Indeed, in one arrangement, a radially outer surface of body 133 may contact and slide along a radially interior surface of ram housing 136. Tab 132 may extend radially outward from body 133 of ram 130, through ram housing 136 (e.g., via a slot 137 (FIG. 2C) or other opening in ram housing 136). Tab 132 may include any suitable features, such as a hole for facilitating a grip of an operator. For example, such a slot or opening may extend along a length of ram housing 136 such that manipulation of tab 132 in an axial direction will cause tab 132 to translate relative to the slot or opening, thereby resulting in advancement and retraction of ram 130 relative to ram housing, handle 112, and shaft 114. In other words, a length of the slot or opening of ram housing 136 may define a stroke length of ram 130. In some arrangements, the opening or slot may extend through a proximal-most end of ram housing 136 such that tab 132, and consequently, ram 130, may be withdrawn proximally from device 100. In other arrangements, the slot or opening may terminate at a location proximal of the proximal-most end of ram housing 136 such that the proximal end of the slot or opening forms a stop so as to limit further axial withdrawal of ram 130. In other words, in such an arrangement, ram 130 may not be removed from ram housing 136 and device 100.

Ram 130 may extend through a housing 152 of handle 112, parallel to or coaxial with a central longitudinal axis Z of shaft 114. Ram 130 may pass through handle 112 without interfering with other components housed in handle 112 (e.g., conduits, wires, actuators, generators, etc.). For example, a vapor generator 160 may be disposed within a grip 154 of handle 112. The vapor generator 160 may be in fluid communication with a lumen (not shown) of a needle 172 extending through shaft 114, and with one or more openings at or near a distal end of the needle 172. For example, a flexible tube (not shown) may be attached to and extend from vapor generator 160 to the proximal end of needle 172. A fluid connector 164 (FIG. 2C) may provide connections between/among sources and/or conduits of fluids (e.g., fluids that may provide flushing, cooling, and/or vapor). For example, fluid connector 164 may include two legs 165, 166 for receiving tubing for providing fluids for flushing, cooling, and/or vapor ablation. Ram 130 may extend longitudinally through fluid connector 164. One or more seal(s) (e.g., O-ring seals) may be utilized between fluid connector 164, ram 130, and/or other tubing of handle 112 to prevent or inhibit fluid from leaking. Ram 130 may pass through an actuator 162, such as a magnetic actuator (e.g., a solenoid actuator) or other type of actuator, for actuating the vapor delivery needle. For example, as shown in FIG. 2C, actuator 162 may define a central opening, through which ram 130 may pass. Ram 130 may further extend into shaft 114 such that axial advancement of ram 130 will result in a transition of shaft 114 from the first, flexible configuration (FIG. 2A) to the second, rigid configuration (FIG. 2B).

A position of ram 130 within shaft 114 may depend upon whether ram 130 is in the first (flexible) configuration of FIG. 2A or the second (rigid) configuration of FIG. 2B. In the first configuration, ram 130 may be in a first, proximal configuration. In the second configuration, ram 130 may be in a second, distal configuration. In the first (flexible) configuration, a distal-most end of ram 130 may be disposed at or near a proximal end of distal portion 124. In the second (rigid) configuration, the distal-most of ram 130 may be disposed at or near a distal end of distal portion 124/at or near a proximal end of distal tip 116. In the first (flexible) configuration, a proximal-most end of ram 130 may be disposed at or near a proximal end of ram housing 136. In the second (rigid) configuration, the proximal-most end of ram 130 may be disposed at or near a distal end of ram housing 136, such that tab 132 abuts the housing 152. As discussed above with respect to FIGS. 1A and 1B, ram 130, tab 132, and/or ram housing 136 may include one or more locks, having any of the properties discussed above. The locks may serve to retain ram 130 in a predefined position (e.g., in the first or second configurations). Additionally or alternatively, locks may facilitate retaining ram 130 at positions between the positions in the first and second configurations. As discussed above with respect to FIGS. 1A and 1B, such intermediate positions of ram 130 may allow an operator to choose to make more or less of shaft 14 flexible.

Device 100 may be operated similarly to device 10, discussed above, to perform a medical method. An operator may insert distal tip 116 of shaft 114 into a urethra of a subject, and advance shaft 114 distally into the urethra. After shaft 114 is positioned such that distal tip 116 is within the prostatic urethra and in a desired location to deliver treatment, the operator may transition the device 100 to the second, rigid configuration. To transition device 100 to the second configuration, the operator may slide tab 132 and the ram 130 distally, relative to ram housing 136, handle 112, and shaft 114. In the second configuration, tab 132 may abut a proximal end of housing 152 of handle 112, such that housing 152 acts as a stop for tab 132 (and the ram 130). The distal end of the ram 130 may extend to or near to distal tip 116 and a distal end of distal portion 124. The ram 130 may stiffen distal portion 124, such that it becomes rigid. With shaft 114 in the second configuration, the operator may deliver a treatment to the subject. For example, the user may depress button 142 in order to deploy a vapor delivery needle and button 144 in order to transurethrally deliver vapor to a prostate of the subject. An operator also may transition device 100 from the second (rigid) configuration to a first (flexible) configuration during a procedure. For example, the operator may move or otherwise slide tab 132 and ram 130 proximally, relative to ram housing 136, handle 112, and shaft 114. The operator may then move a position of distal tip 116, deliver further treatment, and/or withdraw shaft 114 from the body lumen.

Thus, device 100 may provide for a desired flexibility during insertion, while providing a stiffness that is familiar to operators who have used existing devices during delivery of the treatment. An operator may also opt to operate device 100 only in the second configuration. An operator may prefer to insert shaft 114 into a body lumen with shaft 114 in a rigid configuration. For example, the operator may prefer to use the rigid configuration due to operator preference, an anatomy of the subject, a type of treatment, or any other reason. Device 100 may thus provide adaptability in use, such that different operators may use device 100 subject to their own preferences. Device 100 thus may help institutions to avoid needing to buy multiple different types of devices to satisfy the preferences of different operators.

FIG. 3 depicts a portion of a shaft 214, including a distal portion 224 and a proximal portion 222. Any of the features of shaft 214 may be used in conjunction with either of devices 10 or 100. Components of shaft 214 may have any of the properties discussed above, for shafts 14, 114. An outer, flexible sheath 226 may surround other components of distal portion 224 of shaft 214. Proximal portion 222 may be relatively more rigid than distal portion 224, as discussed above with respect to proximal portions 22,122. Shaft 214 may include a distal tip 216, which may have any of the features of distal tips 16, 116, above. A needle 270 may extend through a needle lumen 272 of shaft 214, which may act as an insulation barrier to protect needle 270 from fluid (e.g., flushing fluid) that flows through shaft 214. Needle 270 may include any suitable materials, such as, for example, Ultem®, polysulfone, or Nitinol. As discussed above, needle 270 may be movable proximally and distally relative to shaft 214, in order to transition needle 270 from a first configuration (depicted in FIG. 3), in which a distal tip of needle 270 is within distal tip 216 (or another portion of shaft 214) and a second configuration (not shown), in which a distal tip of needle 270 extends outside of distal tip 216. For example, in the second configuration, a distal portion of needle 270 may extend radially outward from shaft 214. In the second configuration of needle 270, vapor may be delivered through openings at or near a distal tip of needle 270. Wires, cables, conduits, and the like may also extend through lumen 272 or through other portions of shaft 214, between a handle (such as handles 12 or 112) and distal tip 216. As discussed above for shafts 14 and 114, components of distal portion 224 of shaft 214 may be flexible and may be constructed from any of the materials discussed above, with respect to FIG. 1.

A rigid ram 230 may extend through shaft 214. At least portions of ram 230 may be more rigid than an outer sheath of distal portion 224 of shaft 214. Ram 230 may have any of the properties of the rams discussed above with respect to FIGS. 1 and 2. As shown in FIG. 3, a first portion 234 of ram 230 may have a substantially cylindrical, rod-like or tubular shape. A second portion 238 of ram 230, distal of first portion 234, may have a cross-sectional shape (perpendicular to a longitudinal axis of ram 230) that is substantially a segment of a circle or an arc of a circle. Second portion 238 may have a solid cross-section (e.g., a cross section that has an approximately D-shape and is filled in), perpendicular to a longitudinal axis of ram 230. Alternatively, second portion 238 may have an approximately “C” shaped or arc-shaped cross-section, perpendicular to the longitudinal axis of ram 230. A curved surface of second portion 238 may face radially outward, relative to a central longitudinal axis of shaft 214. A shape of the curved surface of second portion 238 may complement an inner surface of sheath 226. In configurations in which second portion 238 has a solid cross-section, second portion 238 may have an approximately planar surface facing in a radially inward direction toward the central longitudinal axis of shaft 214. In configurations in which second portion 238 has an approximately “C” shaped cross-section, ends of the “C” shape may face radially inward, toward the central longitudinal axis of shaft 214. Compared to first portion 234, second portion 238 may have a larger radius of curvature and may have a greater width, perpendicular to the longitudinal axis of ram 230.

Other, more proximal portions of ram 230, not shown in FIG. 3, may have a uniform cross-sectional shape (such as a shape of first portion 234) or may have a varying cross-sectional shape. In alternatives, portions of ram 230 may be flexible. For example, first portion 234 may be flexible, while second portion 238 may be rigid. In such alternatives, first portion 234 and/or more proximal portions of ram 230 may function similarly to an actuation wire, which may be used to move second portion 238 proximally and/or distally. Proximal/distal movement of ram 230 is discussed in further detail below. Second portion 238 may be approximately as rigid as a sheath of proximal portion 222 of shaft 214 (having any of the qualities of proximal portions 22, 122).

A size of second portion 238 (e.g., its relatively larger size as compared to first portion 234) may facilitate stiffening of distal portion 224 of shaft 214. For example, second portion 238 may occupy a large enough area within an interior of sheath 226 and/or extend around a sufficient portion of a circumference of sheath 226 to provide a stiffening of distal portion 224. A curved surface of second portion 238 may be near to an inner surface of sheath 226 or may abut the inner surface of sheath 226, to provide stiffening. It may be beneficial to have first portion 234 occupy a smaller cross-sectional space, to provide more room for other components to pass through a proximal portion of shaft 214. The larger size of second portion 238 may also result in second portion 238 being relatively stiffer than first portion 234. The larger size of second portion 238 may prevent inadvertent bending of ram 230 within shaft 214.

A third portion 236 of ram 230 may extend between first portion 234 and second portion 238. Third portion 236 may remain within proximal portion 222 of shaft 214 in any configuration of ram 230 (discussed in further detail below). Third portion 236 may have any of the properties of second portion 238 and may additionally include the features described below. Third portion 236 may include a guide 237 may protrude radially inwardly toward the central longitudinal axis of shaft 214, from a radially inner surface of third portion 236 (e.g., from a planar surface of third portion 236). Guide 237 may include two arms. Radially outer surfaces of the arms may be curved. A radius of curvature of the outer surfaces of the arm may be substantially the same as a radius of curvature of the radially-outer, curved surface of third portion 236. Radially inner surfaces of the arms of guide 237 may be approximately planar. Alternatively, radially inner surfaces of the arms of guide 237 may be curved. For example, a curved radially inner surface of the arms of guide 237 may complement an outer surface of needle lumen 272. Needle lumen 272 may be received between the arms of guide 237. The arms of guide 237 may be movable (e.g., slidable) relative to needle lumen 272. Ram 230, including first portion 234, second portion 238, guide 237, and distal-most end 239, may be formed of a single, integral piece (i.e., may be a monolithic structure) or may be formed of separate components that are joined together.

Ram 230 may be movable (e.g., slidable) proximally and distally relative to sheath 226 and other components of shaft 214. In a first configuration (not shown), a distal-most end 239 of ram 230 may be proximal of distal portion 224 of shaft 214. Because distal portion 224 may be flexible, distal portion 224 may bend (e.g., passively or actively, via articulation mechanisms) when ram 230 is in the first configuration and does not extend into distal portion 224. In a second configuration (shown in FIG. 3), ram 230 (e.g., second portion 238 of ram 230) may extend into distal portion 224. A distal-most end 239 of ram 230 may near distal tip 216. For example, as shown in FIG. 3, distal-most end 239 of ram 230 may abut or may be near to a housing 274 for components (e.g., camera, lighting, circuitry, conduits for fluids (e.g., flushing fluids), a lumen for receiving needle 270, etc.) of distal tip 216. A proximal face of housing 274 may serve as a stop for distal-most end 239 of ram 230. Because at least second portion 238 of ram 230 may be rigid, shaft 214 may be rigid/unbendable in the second configuration.

As ram 230 moves proximally and/or distally, needle lumen 272 may remain within the arms of guide 237. Guide 237 may retain both ram 230 and needle lumen 272 in their respective positions within sheath 226. For example, guide 237 may maintain a gap between a surface of ram 230 and a surface of needle lumen 272. Guide 237 may assist in maintaining a position of second portion 238 near or contacting an inner surface of sheath 226, to provide for a desired stiffness of distal portion 224. Third portion 236 may remain within proximal portion 222 of shaft 214 in both the first and the second configurations. Because an outer surface shape of third portion 236 may be very similar to an inner surface shape of sheath 226 (e.g., an outer diameter of third portion 236 may be approximately the same as an inner diameter of sheath 226), very little play (e.g., non-axial movement) may be permitted between third portion 236 and sheath 226. Ram 230 may function similarly to a cantilever beam fixed on one end, with a fixed end being comparable to third portion 236, and the beam being comparable to second portion 234. Third portion 236 may thus enhance rigidity of shaft 214 when ram 230 is in the second configuration.

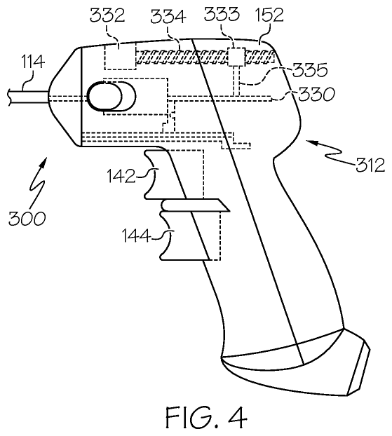

FIGS. 4-6 depict alternative devices having alternative actuation mechanisms. Except as where specified below, the devices of FIGS. 4-6 may incorporate any features of the devices above, including the features of the rams discussed above. Although FIGS. 4-6 may omit certain features of the handles discussed above for ease of illustration of the elements differing from the devices discussed above, it will be appreciated that the handles of FIGS. 4-6 may include any of the features discussed above. Where feasible, like reference numbers are used to denote like features.

FIG. 4 depicts a device 300 having a handle 312. Device 300 may include a ram 330, which may have any of the features of the rams discussed above, except as specified herein. Handle 312 may have a housing 152. A motor 332 (e.g., a micro-motor), which may be operative to rotate a lead screw 334, may be positioned within housing 152. Except for rotating, lead screw 334 may be prevented from moving in an axial direction of lead screw 334. Lead screw 334 may extend through housing 152 of handle 312 in a direction that is approximately parallel to a longitudinal axis of shaft 114 and/or ram 330. Lead screw 334 may include a plurality of threads. A nut 333 may be positioned about lead screw 334. As lead screw 334 rotates in a first direction, nut 333 may move in a first direction (e.g., distally, toward shaft 114). As lead screw 334 rotates in a second direction, opposite to the first direction, nut 333 may move in a second direction, opposite to the first direction (e.g., proximally, away from shaft 114). Lead screw 334 may be coupled to ram 330 via a truss 335. Truss 335 may extend transversely to lead screw 334 and ram 330. For example, truss 335 may be approximately perpendicular to lead screw 334 and ram 330. Truss 335 may be fixed relative to both lead screw 334 and ram 330.

As motor 332 rotates lead screw 334 in the first direction, nut 333 may travel distally along lead screw 334. Nut 333 may move truss 335 distally, as truss 335 is fixed to nut 333. Truss 335 may, in turn, move ram 330 distally, as truss 335 is fixed to ram 330. Ram 330 may eventually move into a position in which ram 330 is fully extended distally (e.g., a position of ram 230 in FIG. 3). As discussed above with respect to FIGS. 1A-3, a distal end of shaft 114 may have a rigid configuration when ram 330 is extended distally. As motor 332 rotates lead screw 334 in the second direction, nut 333 may travel proximally along lead screw 334. Nut 333 may move truss 335 proximally, as truss 335 is fixed to nut 333. Truss 335 may, in turn, move ram 330 proximally, as truss 335 is fixed to ram 330. Ram 330 may eventually move into a position in which ram 330 is fully retracted proximally. As discussed above with respect to FIGS. 1A-3, a distal end of shaft 114 may be relatively more flexible when ram 330 is in a proximal position than when ram 330 is extended distally.

Any suitable actuator may be utilized in order to actuate motor 332 and, in turn, move ram 330 proximally or distally. In some examples, one of buttons 142, 144 may actuate motor 332, in addition to or in alternative to the functions described above for buttons 42, 44, 142, 144. Alternatively, handle 312 may include an additional actuator (e.g., button, switch, lever, knob, or other actuator, not shown) for actuating motor 332 and moving ram 330 proximally or distally.

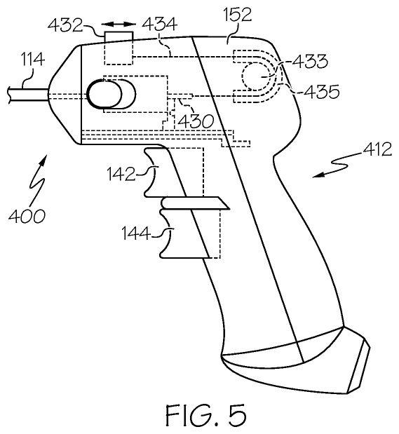

FIG. 5 depicts an alternative device 400 having a handle 412. Device 400 may include a ram 430, which may have any of the features of the rams discussed above, except as specified herein. Within housing 152, proximal end of ram 430 may be fixed to a flexible cable or wire 434. Although the term “wire” may be used herein, it will be appreciated that the disclosure is not limited to wires and includes any flexible structure (e.g., cable, microtube, threads, filaments, etc.) Alternatively, a proximal end of ram 430 may itself be flexible, and wire 434 may be omitted. Wire 434 may be wrapped about a pulley 433. An end of wire 434 that is not fixed to ram 430 may be fixed to an actuator 432. As shown in FIG. 5, actuator 432 may include a slider. However, actuator 432 is not so limited and may include levers, knobs, or the like. Actuator 432 may pass through an opening of housing 152 (e.g., a slot in housing 152). An operator may contact a portion of actuator 432 that is external to housing 152. Additionally or alternatively, one or more of buttons 142 or 144 may be configured to actuate actuator 432 upon a press of button(s) 142/144.

In the example depicted in FIG. 5, wire 434 may extend proximally from ram 430 toward pulley 433, approximately coaxial with ram 430. Pulley 433 may be positioned near a proximal end of housing 152. Wire 434 may be attached to actuator 432 in such a matter that, after wrapping around pulley 433, wire 434 extends approximately parallel to ram 430. However, alternative arrangements are included within the scope of the disclosure. A sleeve 435 (e.g., a tube, channel, or guide) may be positioned about pulley 433. Wire 434 may pass through or about sleeve 435, in order to retain wire 434 in a desired position about pulley 433 and prevent wire 434 from slipping off of pulley 433.

In the example in which actuator 432 is a slider, actuator 432 may be moved proximally or distally, in the directions shown by the arrows of FIG. 5. As actuator 432 is moved proximally, an end of wire 434 attached to ram 430 may be moved distally. Distal movement of the end of wire 434 attached to ram 430 may, in turn, exert a distal force on ram 430, causing ram 430 to move to distally. Ram 430 may eventually transition to a configuration in which ram 430 is extended fully in a distal direction. As discussed above with respect to FIGS. 1A-3, a distal end of shaft 114 may have a rigid configuration when ram 430 is extended distally. As actuator 432 is moved distally, an end of wire 434 that is attached to ram 430 may move proximally. Wire 434 may thus pull ram 430 proximally. Ram 430 may eventually move into a position in which ram 430 is fully retracted proximally. As discussed above with respect to FIGS. 1A-3, a distal end of shaft 114 may be relatively more flexible when ram 430 is in a proximal position than when ram 430 is extended distally.

A range of motion of actuator 432 may correspond to configurations in which ram 430 is moved fully proximally or fully distally. For example, when actuator 432 is moved as distally as possible (e.g., to a distal end of a slot in housing 152), ram 430 may be fully retracted proximally. When actuator 432 is moved as proximally as possible (e.g., to a proximal end of a slot in housing 152), ram 430 may be fully extended distally.

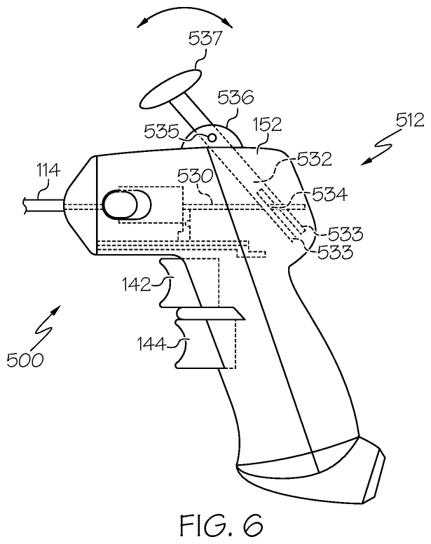

FIG. 6 depicts a device 500 having a handle 512. Device 500 may include a ram 530, which may have any of the features of the rams discussed above, except as specified herein. Within housing 152, ram 530 may be rotatably coupled to a lever 532 at a pivot point 534. For example, as shown in FIG. 6, lever 532 may include two legs 533. A proximal end of ram 530 may extend between legs 533 and may be coupled to legs 533 via, e.g., a pin, rivet, or other coupling device. Ram 530 may be restricted to axial movement (i.e., may not rotate or move laterally). For example, support structures (not shown) may limit ram 530 to movements along a longitudinal axis of ram 530. A handle 537 of lever 532 may extend through an opening of housing 152 (e.g., a slot of housing 152). Lever 532 may be rotatably coupled to a protrusion 536 that extends from housing 152 via a pivot 535 (e.g., a pin, rivet, or other coupling device).

An operator may rotate handle 537 about an axis of pivot 535, in directions shown by the arrow of FIG. 6. As handle 537 is rotated in a clockwise direction of FIG. 6, lever 532 may exert a distal force on ram 530, causing ram 530 to move to distally. Ram 530 may eventually transition to a configuration in which ram 530 is extended fully in a distal direction. As discussed above with respect to FIGS. 1A-3, a distal end of shaft 114 may have a rigid configuration when ram 530 is extended distally. Lever 532, including legs 533, may pivot/rotate relative to ram 530. A space in between legs 533 may facilitate rotation of lever 532 while maintaining only axial movement of ram 530. As handle 537 is rotated in a counter-clockwise direction, lever 532 may exert a proximal force on ram 530, causing ram 530 to move proximally. Ram 530 may eventually move into a position in which ram 530 is fully retracted proximally. As discussed above with respect to FIGS. 1A-3, a distal end of shaft 114 may be relatively more flexible when ram 530 is in a proximal position than when ram 530 is extended distally.

A range of motion of lever 532 may correspond to configurations in which ram 530 is moved fully proximally or fully distally. For example, when lever 532 is moved fully counter-clockwise (e.g., until handle 537 contacts housing 152 or until lever 532 reaches an end of a slot in housing 152), ram 430 may be fully retracted proximally. When lever 532 is moved as far clockwise as possible (e.g., until handle 537 contacts housing 152 or until lever 532 reaches an end of a slot in housing 152), ram 430 may be fully extended distally.

The above-described mechanisms for actuating the rams discussed above are merely exemplary. It will be appreciated that other mechanisms, including a slide on an outside of a shaft (e.g., shaft 14, 114) that is magnetically coupled to any of the rams discussed above, linkages, hydraulic/pneumatic internal cylinders, or solenoid actuators may be used to actuate the rams discussed above.

While principles of the present disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a handle including:
a grip portion;
a body portion; and
a ram housing extending proximally of the body portion;
a shaft having a first, proximal portion and a second, distal portion, wherein the first portion of the shaft includes a first outer sheath, wherein the second portion of the shaft includes a second outer sheath, and wherein the first outer sheath is more rigid than the second outer sheath; and
a rigid ram extending through at least a portion of the body portion and at least a portion of the shaft, wherein the ram is configured to be transitioned from a first, proximal configuration, in which the second portion of the shaft is flexible, and a second, distal configuration, in which the second portion of the shaft is rigid.

2. The medical device of claim 1, wherein a proximal end of the ram protrudes from a proximal end of the body portion of the handle, and wherein the ram is transitioned from the first configuration to the second configuration by moving the proximal end of the ram distally.

3. The medical device of claim 2, wherein the proximal end of the ram includes a tab.

4. The medical device of claim 3, wherein the proximal end of the ram is received within the ram housing, and wherein the tab extends through a slot in the ram housing.

5. The medical device of claim 3, wherein, in the second configuration, the tab abuts a proximal surface of the body portion of the handle.

6. The medical device of claim 2, wherein an axis of the grip portion is transverse to a longitudinal axis of the first portion of the shaft.

7. The medical device of claim 1, wherein, in the first configuration, a distal-most end of the ram is proximal of at least a portion of the second portion of the shaft.

8. The medical device of claim 1, wherein the first outer sheath is approximately as rigid as a distal portion of the ram.

9. The medical device of claim 1, wherein at least a portion of the ram has a cross-sectional shape that is approximately a segment of a circle or approximately an arc of a circle.

10. The medical device of claim 9, wherein the at least the portion of the ram includes a curved, radially outward-facing surface, and an approximately planar, radially inward-facing surface.

11. The medical device of claim 10, wherein a guide, having a first arm and a second arm, extends radially inwardly from the approximately planar, radially inward-facing surface.

12. The medical device of claim 11, wherein a lumen is received between the first arm and the second arm.

13. The medical device of claim 12, wherein a needle is received within the lumen.

14. The medical device of claim 13, wherein a distal opening of the needle is in fluid communication with a vapor generator in the handle.

15. The medical device of claim 1, wherein the second portion of the shaft includes a flexible, tubular sheath.

16. A medical device, comprising:
a handle;
a shaft having a first portion, having a rigid outer sheath, and a second portion, having a flexible outer sheath, wherein each of the first portion and the second portion of the shaft is configured to be inserted into a body lumen of a subject; and
a rigid ram that is configured to be transitioned from a first configuration, in which a distal-most end of the ram is proximal of the second portion of the shaft, to a second configuration, in which the distal-most end of the ram is within the second portion of the shaft, wherein a proximal end of the ram protrudes from a proximal end of the handle, and wherein the ram is transitioned from the first configuration to the second configuration by moving the proximal end of the ram in a distal direction.

17. The medical device of claim 16, wherein the second portion of the shaft is bendable in the first configuration, and wherein the second portion of the shaft is rigid in the second configuration.

18. The medical device of claim 16, wherein the proximal end of the ram is received within a ram housing, and wherein a tab of the proximal end of the ram extends through a slot in the ram housing.

19. A medical method, the method comprising:
inserting a distal end of a shaft of a medical device into a body lumen of a subject;
navigating the distal end of the shaft to a treatment location;
after navigating the distal end of the shaft to the treatment location, moving a ram distally from a first configuration to a second configuration, wherein a portion of shaft is bendable in the first configuration, and wherein the portion of the shaft is rigid in the second configuration;
in the second configuration, extending a needle from a distal tip of the shaft; and
delivering a vapor from the needle.

20. The medical method of claim 19, wherein the body lumen is a urethra, and wherein the vapor is configured to ablate at least a portion of a prostate of the subject.
